(19) United States
(12) Patent Application Publication
(10) Pub. No.: US 2007/0298017 A1
(43) Pub. Date: Dec. 27, 2007

(54) Composition for Treating Baldness with Stem Cell Derived From Umbilical Cord Blood

(76) Inventor: Hoon Han, Gyeonggi-do (KR)

Correspondence Address: VENABLE LLP, P.O. BOX 34385, WASHINGTON, DC, 20043-9998, US

(21) Appl. No.: 11/794,210

(22) PCT Filed: Dec. 13, 2005
(86) PCT No.: PCT/KR05/04237

§ 371 Date: Jun. 26, 2007

(30) Foreign Application Priority Data
Dec. 30, 2004 (KR) 10-2004-0116639

(51) Int. Cl. A61K 35/44; A61P 17/14

(52) U.S. Cl. 424/93.7

(57) ABSTRACT

Provided is a therapeutic technique for treating baldness, using an umbilical cord blood-derived stem cell. Transplantation of a composition for treating baldness into a bald area of a patient can make great contributions to treatment for baldness, wherein the composition comprises stem cells isolated and cultured from umbilical cord blood in which 6 HLA (Human Leukocyte Antigen) are identical with a patient, or one or two HLA are not identical with the patient.

### TECHNICAL FIELD

The present invention relates to a technique for treating baldness, using an umbilical cord blood-derived stem cell.

### BACKGROUND ART

Baldness is a common condition characterized by partial or complete loss or thinning of hair on the scalp due to hair falling out. At present, there is still no method known that is capable of completely reversing and essentially curing this condition. Loss of hair due to a disorder of hair growth is scientifically known as alopecia. Alopecia refers to a clinical condition in which all or almost all of the hairs are completely or partially lost. The exact mechanism for the disorder of hair growth is not still fully understood, but it is speculated that alopecia occurs from dysfunction or disorder in relationship or interaction between hair follicle development and a hair cycle.

The hair cycle is divided into three phases as follows: the growing phase (anagen), the transitional phase (catagen) and the resting phase (telogen). Firstly, the anagen phase of a new hair starts at the moment it begins to actively grow. At that time there is very active differentiation of hair matrix cells in anagen hair follicles. Secondly, the catagen phase is the intermediate phase of the hair growth cycle. During the catagen phase, hair matrix cells cease hair production and the hair becomes a specific shape, called a club hair resembling a club. The last one, i.e. telogen phase is a resting phase during which hair follicles completely stop their activity and shrink. FIG. 1 is a schematic view showing a hair growth cycle.

If abnormal conditions are caused or introduced in the hair growth cycle as shown in FIG. 1, new hair may not be regenerated upon alopecia, and thus this means that hair stem cells, involved in hair growth and production, no longer function. Non-functioning of the hair stem cells in the hair growth process results in non-functioning of hair papillae beneath hair follicles, and as a result, a process of hair falling out progressively worsens, thereby making it difficult to achieve new hair growth from this time on.

A great deal of interest has also been focused on stem cells related to the hair hitherto and considerable research and studies have been made on animal models. Reference may be made to a variety of published scientific articles and journals. For example, it was recently reported that hair graying is triggered by incomplete melanocyte stem cell maintenance (Sciencexpress, 23 Dec. 2004). In addition, another article has reported that adult mice have multipotent stem cells which are related to morphogenetic signals involved in formation of multiple hair follicles (Cell, Vol 104, 233-245, Jan. 26, 2001). FIG. 2 is a schematic view showing fate of a multipotent stem cell.

According to other articles, it was also reported that follicular epithelial stem cells are present in hair follicle stem cells and these epithelial stem cells function to modulate the hair cycle (JID Symposium Proceedings 8:28-38, 2003). In addition, it was reported that interrelationship between the epithelium and mesenchyme is a crucial factor in formation of hair follicles. Further, a certain article has reported an experiment on whether hair growth can be triggered by expressing a specific gene related to the dermal papilla in a nude mouse (Proc. Natl, Acad. Sci. USA 96. 1999). FIG. 3 is a series of photographs showing effects of expression of the dermal papilla-related specific gene in the nude mouse. a: 4 weeks after expression of the specific gene. b: When the specific gene was not expressed. c: 4 months after expression of the specific gene. Meanwhile, FIG. 4 is a photograph showing the results of expression of a gene related to hair follicle growth (Hox gene). Left: Normal mouse. Right: Transformed mouse in which the Hox gene was overexpressed (Naturwissenschaften 90: 193-211, 2003).

Unfortunately, there is yet no therapeutically effective method capable of fundamentally preventing or treating alopecia, and alternative methods, which are currently available by modern medical techniques, include, for example surgical operations such as hair transplantation and scalp plastic surgery, and drug therapies. Hair transplantation is one of the permanent solutions to balding, in which hair roots are removed from the donor region still having normal functions along the back and sides of the head, and transplanted into the bald region where hair roots were dead. Surgical operations such as scalp plastic surgery include for example scalp reduction surgery, scalp flap surgery and tissue extension or tissue expansion. Examples of drug therapies include use of Minoxidil (a hypotensive drug) and Propecia (a prostate gland shrinking medication). Propecia and Minoxidil are the only two medications that are FDA approved for hair re-growth. However, discontinuation of drug administration results in substantially no effects on treatment for hair loss. Therefore, the above-mentioned treatments are merely temporary means rather than permanent ones.

That is, unlike normal hair growth, alopecia is a condition where the ability to regenerate and produce new hair is lost, and thus it seems impossible to regenerate dermal papillae of the hair follicles. However, it is considered that if umbilical cord blood-derived stem cells, in which genetic synthesis and knowledge of relevant characteristics thereof were known in advance, are used in treatment for alopecia, it will be possible to solve problems associated with immune rejection and it will provide better therapeutic effects and results than use of patients own cells which may already have genetic defects.

In general, stem cells refer to primitive cells having a self-renewal ability whereby they can undergo continuous proliferation in the immature and undifferentiated state, and a differentiation ability whereby they can differentiate into other specialized cells and tissues. Although it is easy to isolate and cultivate stem cells from bone marrow, there is difficulty in acquisition of the bone marrow and further, at present it is known to be difficult to solve problems associated with immune rejection occurring when transplanting the thus-obtained stem cells to another person. Meanwhile, umbilical cord (neonatal) blood is relatively easy to obtain compared with bone marrow, and also, where great numbers of umbilical cord blood units are secured, it is possible to employ umbilical cord blood stem cells having histocompatibility genes that are identical with or most similar to those of patients and thereby it is possible to solve problems associated with immune rejection.

As discussed above, there is yet no attempt to treat baldness using stem cells throughout the world hitherto, excluding a few animal experiments using mice or the like. Particularly, to the best of our knowledge, there is no case in which baldness patients were treated using the umbilical cord blood-derived stem cells.

### DISCLOSURE OF INVENTION

### Technical Problem

Therefore, the present invention has been made in view of the above problems, and it is an object of the present invention to provide a basis of cell culture and cell transplantation for treating baldness, and particularly provide a therapeutic technique for treating baldness using an umbilical cord blood-derived stem cell.

### Technical Solution

In accordance with an aspect of the present invention, the above and other objects can be accomplished by the provision of a composition for treating baldness, comprising stem cells isolated and cultured from umbilical cord blood in which 6 HLA (Human Leukocyte Antigen) are identical with those of a patient, or one or two HLA are not identical with those of the patient.

Specifically, the composition for treating baldness is preferably a composition for transplanting into a bald area of a patient for treating baldness, comprising umbilical cord blood-derived stem cells obtained by:

diluting umbilical cord blood with an alpha-minimum essential medium (αMEM), followed by centrifugation to harvest monocytes;

isolating CD133-positive cells from the monocytes; and

subjecting the isolated CD133-positive cells into suspension culture in the αMEM containing an antibiotic, an anti-fungal agent, a fetal bovine serum and glutamine.

In accordance with another aspect of the present invention, there is provided a method for treating baldness, comprising:

selecting umbilical cord blood in which 6 HLA (Human Leukocyte Antigen) are identical with those of a patient, or one or two HLA are not identical with those of the patient;

isolating and culturing stem cells from the selected umbilical cord blood; and

transplanting the cultured stem cells into a bald area of a patient.

In the present invention, stem cells are isolated and cultured from the umbilical cord blood selected in a manner that immune rejection does not occur between donor stem cells and the patient to be treated, upon transplanting the stem cells, and the thus obtained stem cells are transplanted to the bald area, thereby treating the baldness patient.

For this purpose, the inventors of the present invention have developed a patient-specific, immune-matched umbilical cord blood-derived cell therapy which enables production of normal hair by transplanting umbilical cord blood-derived stem cells into patients suffering from alopecia who are incapable of producing normal hair due to genetic defects, thereby leading to an activated state of hair stem cells which have already lost their normal functions. Therefore, transplantation of such umbilical cord blood-derived stem cells into the bald area results in production of hair which in turn plays a key role in maintenance and hair color and undergoes self-replication and proliferation, thereby constantly maintaining its own functions. Further, when the umbilical cord blood-derived stem cells are transplanted into the scalp, the stem cells are implanted into various skin appendages and hair bulge regions of hair follicles, undergo self-replication and then propagate via blood vessels to neighboring hair follicles, thus affecting the entire scalp hair. In addition, similar to the fetal period, the umbilical cord blood-derived stem cells differentiate into hair matrix cells and melanocytes beneath hair follicles, thereby resulting in active production of the hair. Further, as a growth period of hair is prolonged, thin vellus hair changes into thick and long terminal hair.

The reason why a cell therapy for curing alopecia can be achieved using the umbilical cord blood-derived stem cells is because hair bulge regions are healthy and intact in terms of their functions even when lower parts of hair follicles have atrophied due to effects of an androgen hormone in male-pattern baldness, and therefore the umbilical cord blood-derived stem cells are adhered thereto, thereby exhibiting their functions. Herein, a critical factor that should be considered in the first place consists in use of the umbilical cord blood-derived stem cell in which HLA is identical with that of patient to be treated. That is, it is possible to solve immune rejection only in the case of using such an umbilical cord blood-derived stem cell having HLA identical with that of the patient. In addition, use of the umbilical cord blood-derived stem cells enables secretion of growth factors and cytokines necessary for hair growth, and as a result, leads to activation of the existing hair growth phase, thereby minimizing a hair loss rate or being differentiated into hair stem cells, assisting in formation of hair follicles which in turn allows for hair growth.

In conventional arts, there is no method for treating baldness using the umbilical cord blood-derived stem cell. Further, although a method for treating baldness using patient's own bone marrow is theoretically discussed, it is practically not easy to obtain bone marrow. Therefore, it can be said that use of stem cells contained in the neonatal umbilical cord blood will provide a more reliable method for treating baldness than use of patient's own stem cells which may already have genetic defects.

### DESCRIPTION OF THE DRAWINGS

The above and other objects, features and other advantages of the present invention will be more clearly understood from the following detailed description taken in conjunction with the accompanying drawings, in which:

FIG. 1 is a schematic view showing a hair growth cycle;

FIG. 2 is a schematic view showing fate of a multipotent stem cell;

FIG. 3 is a set of photographs showing results of expression of a dermal papilla-related specific gene in nude mice. a: 4 weeks after expression of the specific gene. b: When the specific gene was not expressed. c: 4 months after expression of the specific gene;

FIG. 4 is a photograph showing results of expression of a gene related to hair follicle growth (Hox gene). Left: Normal mouse. Right: Transformed mouse in which Hox gene was overexpressed;

FIG. 5 is a series of scope photographs showing the frontal hairline of the head taken after treatment with an umbilical cord blood-derived stem cell in accordance with the present invention. a: Hair of a normal adult. b: Frontal hairline of the head after treatment;

FIG. 6 is a series of photographs showing conditions of the bald area of the scalp, taken before transplantation of umbilical cord blood-derived stem cells obtained in accordance with the present invention, and 17 weeks and 25 weeks after transplantation thereof, respectively. a: Before transplantation. b: 17 weeks after transplantation. c: 25 weeks after transplantation;

FIG. 7 is a series of photographs showing conditions of the bald area of the scalp, taken before transplantation of umbilical cord blood-derived stem cells obtained in accordance with the present invention, and 17 weeks after transplantation thereof, respectively. a: Before transplantation. b: 17 weeks after transplantation; and

FIG. 8 is a series of photographs showing conditions of the bald area of the scalp, taken before transplantation of umbilical cord blood-derived stem cells obtained in accordance with the present invention, and 3 weeks after transplantation thereof, respectively. a: Before transplantation. b: 3 weeks after transplantation.

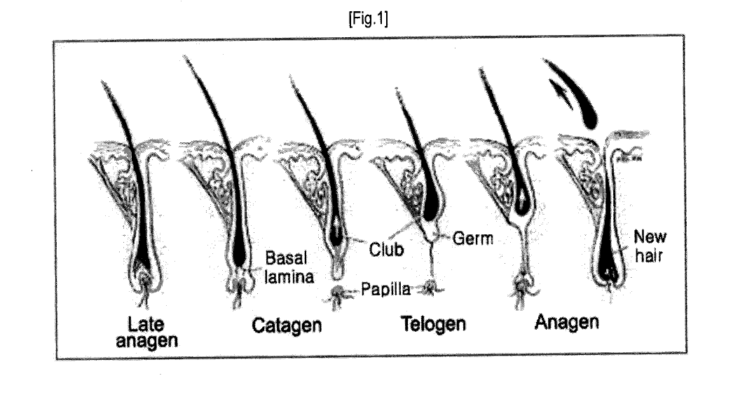

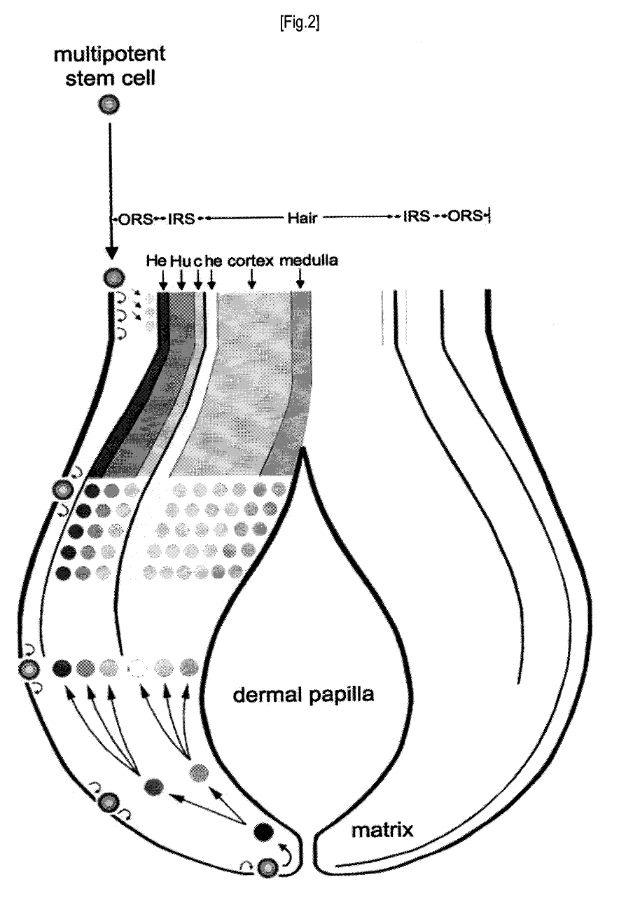

### BEST MODE

Hereinafter, a technique for treating baldness using an umbilical cord blood-derived stem cell in accordance with the present invention will be described in more detail with reference to the following Examples. These examples are provided only for illustrating the present invention and should not be construed as limiting the scope and sprit of the present invention.

### EXAMPLES

### Example 1

### Selection of Umbilical Cord Blood

After determining as to whether 6 Human Leukocyte Antigen (HLA) are identical with those of baldness patients, cryopreserved umbilical cord blood was selected in which 6 HLA were identical with those of patients or one or two HLA were not identical with those of patients.

The human leukocyte antigen (HLA) is an important factor determining acceptance or rejection of the engrafting of the injected cell, when foreign (non-self) cells other than autologous (self) cells are injected into the body. In order to determine histocompatibility between a donor and a recipient, HLA is subjected to examination of the total 6 antigens, each examination of which is based entirely on DNA analysis. Such DNA analysis of 6 antigens is to determine on whether the HLA class I (HLA-A, HLA-B) and class II (HLA-DR) loci are identical with those of a patient to be transplanted.

### Example 2

### Isolation and Culture of Stem Cells from Umbilical Cord Blood

Umbilical cord blood units cryopreserved at −196° C. were placed and immediately thawed in a water bath at 37° C. In order to isolate monocytes from the umbilical cord blood, the umbilical cord blood was diluted with two-fold volume of αMEM (alpha-minimum essential medium, Jeil Biotech Services, Korea) and was centrifuged at 300×g for 10 minutes at room temperature. The separated buffy coat layer was collected, diluted again with two-fold volume of αMEM, overlapped on Ficoll-Hypaque and centrifuged at 300×g for 30 minutes at room temperature.

In isolating monocytes from blood, Ficoll-Hypaque, which is a polymer of Ficoll (sucrose polymer) and Hypaque (sodium ditrizoate), is largely used. Ficoll-Hypaque has a specific gravity of 1.077 g/ml, which is heavier than that of monocytes, but lighter than that of red blood cells, which makes it possible to separate the cells from each other by specific gravity difference therebetween. That is, when blood is placed on Ficoll-Hypaque and centrifuged, monocytes gather on the Ficoll-Hypaque.

Monocytes obtained by such a density gradient centrifugation method were additionally washed twice with a washing αMEM in which additives were not included.

From the thus-obtained monocytes, CD133-positive cells were selectively isolated using an Isolation kit (Miltenyi Bioteck, Germany) as follows: 100 μl of a blocking reagent was added to monocytes so as to remove non-specific bonding, and then homogeneously mixed with 100 μl of a CD133/Microbead to a total volume of 500 μl. The resulting mixture was then cultured at 4° C. for 30 minutes. The culture was added with a ten-fold volume of PBS (D-phosphate buffered saline, Jeil Biotech Services, Korea), centrifuged at 300×g for 10 minutes, and thereafter, PBS was discarded, thereby obtaining the cells adhered to the tube. The cells thus obtained were resuspended in 500 μl of PBS. After the column of Isolation kit was previously washed with 3 ml of a PBS buffer, the resuspended cells were loaded and maintained in the column for more than 15 minutes. The column, after being rinsed with PBS four times, was removed from the kit and then added with an appropriate amount of PBS in a tube, followed by flushing using a plunger, thereby selecting positive cells.

Next, the selected cells were cultured for 5 days in αMEM (1000 U/ml of penicillin G, 1000 μg/ml of streptomycin sulfate, Gibco-BRL) containing 20% fetal bovine serum (FBS, Jeil Biotech Services, Korea), and an anti-fungal agent (0.25 μg/ml amphotericin B) and 2 mM glutamine (Sigma). After five-day culturing, suspended cells were removed from the cultured cell population. When adherent cells were obtained, they were cultured in αMEM having the same composition as a culture medium, with complete replacement of a culture medium at intervals of 2 days.

### Example 3

### Transplantation of Umbilical Cord Blood-Derived Stem Cells

Stem cells (5×10⁷ cells), cultured for two weeks in Example 2, were placed in 2 mL of 0.05% trypsin-EDTA and were reacted at room temperature for 5 min. Then, 3 mL of αMEM was added thereto and the resulting mixture was transferred to a 15 mL test tube and centrifuged at 300×g for 10 min. After centrifugation, a supernatant was discarded and 15 mL of physiological saline (Choong-Wae Pharmaceutical Corporation, Seoul, Korea) was added to the remaining materials in a test tube and were centrifuged at 300×g for 10 min and a supernatant was discarded. This step was repeated thrice. The thus-obtained cells were resuspended in 6 mL of physiological saline, and 2 mL of suspended cells were subcutaneously transplanted into right/left and rear parts of the bald area anesthetized along hairless lines, respectively, using a 26 G syringe needle.

This transplantation method of umbilical cord blood-derived stem cells enables the patient to be immediately discharged from a hospital after cell transplantation is complete. It could be confirmed that hair was produced from 2 weeks after transplantation, depending upon the degree and severity of baldness.

### Example 4

### Transplantation of Umbilical Cord Blood-Derived Stem Cells into Patients and Results Thereof

18 baldness patients (16 males and 2 females) took part in treatment for alopecia using umbilical cord blood-derived stem cells in accordance with the present invention.

(1) Male patient, age of 51 (operation conducted in July of the year 2003)

Before being treated for baldness, hair remained only along the sides and rear area of the head. After performing transplantation operation of umbilical cord blood-derived stem cells, thin hair began to grow from the point of 2 weeks, and 6 months later, it was possible to comb the hair. Therapeutic response by the transplanted stem cells was observed throughout the scalp hair, including initiation of frontal hairline formation and changes of neighboring white hair into black hair. Upon observing on a digital magnification scope, growth of thin hair was confirmed even in the area where no hair was apparently seen by naked eyes.

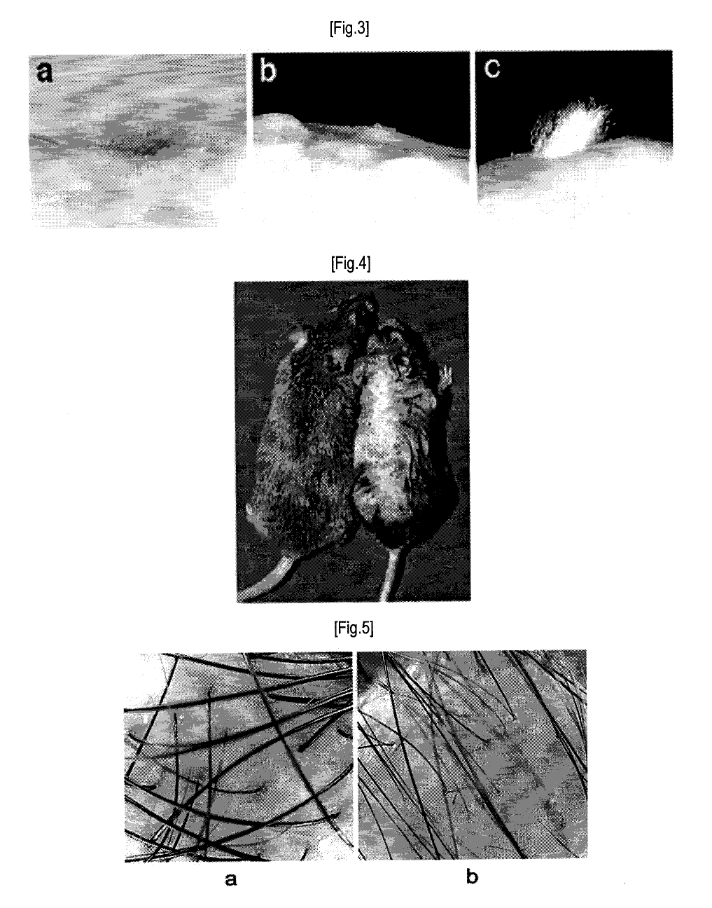

FIG. 5 is a series of scope photographs of the frontal hairline of the head taken after treatment with an umbilical cord blood-derived stem cell in accordance with the present invention, wherein a shows hair of a normal adult and b shows a frontal hairline after treatment.

(2) Male patient, age of 48 (operation conducted at the end of October of the year 2003)

Before receiving the treatment for baldness, the patient suffered baldness having a diameter of about 5 cm in the frontal area of the head. After treatment with umbilical cord blood-derived stem cells, it was observed that hair conditions were recovered close to a normal state.

(3) Male patient, age of 55 (operation conducted in September of the year 2003)

Before performing transplantation operation of umbilical cord blood-derived stem cells, the patient suffered baldness having a diameter of about 15 cm on the vertex of the head. After treatment using the stem cells, hair began to grow from the point of 4 weeks, and the hairline moved up about 4 cm higher by December. Subsequently, a hair density became higher, and new hair was actively produced and growing.

(4) Male patient, age of 51 (operation conducted in September of the year 2003)

Before receiving the treatment for baldness, the patient suffered severe baldness on the crown of the head. After treatment using the stem cells, hair began to grow and proliferate from the point of 3 weeks, and has consequently reached a hair condition close to that of normal people.

(5) Male patient, age of 51 (operation conducted in September of the year 2003)

Before performing transplantation operation of stem cells to treat baldness, the patient suffered partial baldness on the vertex of the head, in conjunction with progression of frontal hair loss in a typical “M”-shaped pattern. After treatment using the stem cells, it was observed that the bald area recovered to normal hair conditions.

FIG. 6 is a series of photographs showing conditions of the bald area of the scalp, taken before transplantation of umbilical cord blood-derived stem cells obtained in accordance with the present invention, and 17 weeks and 25 weeks after transplantation thereof, respectively, wherein a is a photograph taken before transplantation, b is a photograph taken 17 weeks after transplantation, and c is a photograph taken 25 weeks after transplantation.

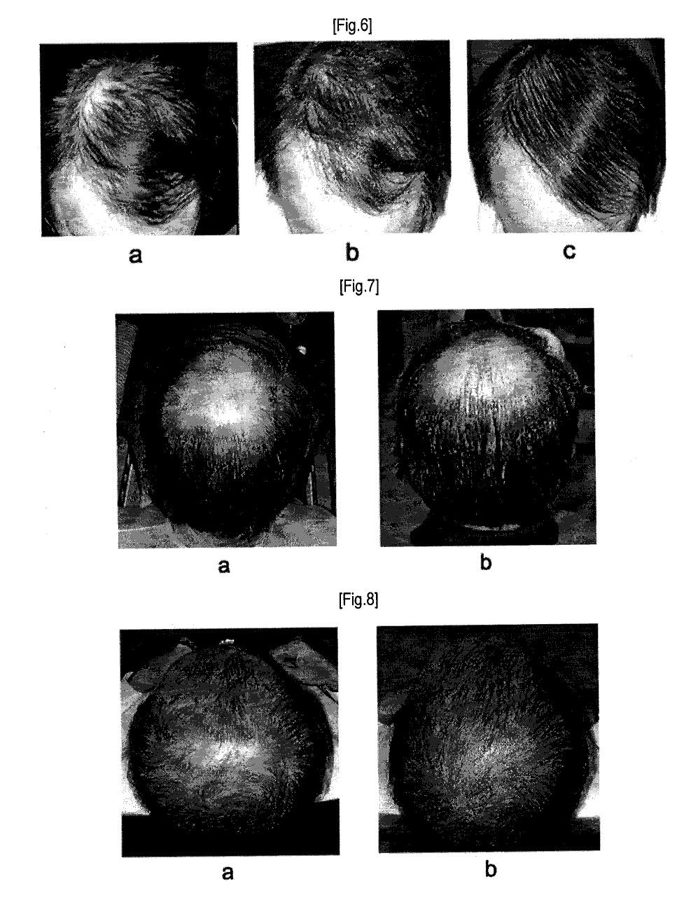

FIG. 7 is a series of photographs showing conditions of the bald area of the scalp, taken before transplantation of umbilical cord blood-derived stem cells obtained in accordance with the present invention, and 17 weeks after transplantation thereof, respectively, wherein a is a photograph taken before transplantation and b is a photograph taken 17 weeks after transplantation.

FIG. 8 is a series of photographs showing conditions of the bald area of the scalp, taken before transplantation of umbilical cord blood-derived stem cells obtained in accordance with the present invention, and 3 weeks after transplantation thereof, respectively, wherein a is a photograph taken before transplantation and b is a photograph taken 3 weeks after transplantation.

As shown in FIGS. 6 through 8, it can be seen that significant therapeutic effects on baldness were exerted from 3 weeks after transplantation of the umbilical cord blood-derived stem cells.

### INDUSTRIAL APPLICABILITY

As described and demonstrated above, in accordance with the method of the present invention, it is anticipated that transplantation of the umbilical cord blood-derived stem cells into a bald area will make great contributions to treatment for baldness taking into consideration the fact that there is no alternative method for curing baldness.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for treating baldness, comprising stem cells isolated and cultured from umbilical cord blood in which 6 HLA (Human Leukocyte Antigen) are identical with those of a patient, or one or two HLA are not identical with those of the patient.

2. A composition for transplanting into a bald area of a patient for treating baldness, comprising umbilical cord blood-derived stem cells obtained by:
diluting umbilical cord blood with an alpha-minimum essential medium (αMEM), followed by centrifugation to harvest monocytes;
isolating CD133-positive cells from the monocytes; and
subjecting the isolated CD133-positive cells into suspension culture in the αMEM containing an antibiotic, an anti-fungal agent, a fetal bovine serum and glutamine.

3. A method for treating baldness, comprising:
selecting umbilical cord blood in which 6 HLA (Human Leukocyte Antigen) are identical with those of a patient, or one or two HLA are not identical with those of the patient;
isolating and culturing stem cells from the selected umbilical cord blood; and
transplanting the cultured stem cells into a bald area of a patient.
